(19) United States
(12) Patent Application Publication
(10) Pub. No.: US 2026/0004937 A1
(43) Pub. Date: Jan. 1, 2026

(54) SYSTEMS AND METHODS TO PROCESS ELECTRONIC IMAGES TO PREDICT PROGRESSION AND REGRESSION

(71) Applicant: Heartflow, Inc., Mountain View, CA (US)

(72) Inventors: Michiel SCHAAP, Oegstgeest (NL); Adam UPDEPAC, Alamo, CA (US); Andreas SCHUH, Cambridge (GB); Peter Kersten PETERSEN, Palo Alto, CA (US); Matthew SINCLAIR, London (GB); Nan XIAO, San Jose, CA (US); Sabrina LYNCH, Cupertino, CA (US); Samuel GERBER, Fayetteville, WV (US); Souma SENGUPTA, Cupertino, CA (US); Timothy A. FONTE, San Francisco, CA (US)

(21) Appl. No.: 19/255,328

(22) Filed: Jun. 30, 2025

Related U.S. Application Data
(60) Provisional application No. 63/666,366, filed on Jul. 1, 2024.

(51) Int. Cl. G16H 50/30; G06N 20/00; G16H 10/60; G16H 50/20; G16H 50/70

(52) U.S. Cl. CPC G16H 50/30; G06N 20/00; G16H 10/60; G16H 50/20; G16H 50/70

(57) ABSTRACT

A computer-implemented method for predicting cardiovascular disease risk, the method including: receiving a first patient history data comprising imaging data and/or non-imaging data for a patient at a first time point; selecting prediction report parameters defining a type of cardiovascular event and a risk prediction time scale; processing the first patient history data using a trained machine learning model configured to predict disease onset, progression, and/or regression over time, wherein the trained machine learning model is trained using patient subsets created based on patient history characteristics and outcomes; generating a risk prediction for the selected type of cardiovascular event over the selected risk prediction time scale based on the processed first patient history data; generating a risk prediction report based on the risk prediction; and outputting the risk prediction report.

### CROSS REFERENCE TO RELATED APPLICATIONS

This application claims priority to U.S. Provisional Application No. 63/666,366, filed on Jul. 1, 2024, the entire disclosure of which is hereby incorporated by reference in its entirety.

### TECHNICAL FIELD

The present disclosure relates to medical imaging and machine learning systems for cardiovascular disease assessment, and more particularly to systems and methods for processing electronic images and patient data to predict cardiovascular disease risk, progression, and regression using artificial intelligence-based analysis.

### INTRODUCTION

Risk assessment to optimize preventive strategies based on the absolute short- and long-term risk of an individual comprises the cornerstone of cardiovascular disease (CVD) prevention. Cardiovascular disease (CVD) clinical risk estimation models, such as the Framingham Risk Score and the Pooled Cohort Equations, estimate the risk of a first CVD event using routinely collected risk factors-age, blood pressure, diabetes, smoking status, and cholesterol levels. These risk estimates can be used to inform shared decision-making with patients regarding lifestyle improvements, risk factor control, and medical therapy.

Risk factor-based models fail to accurately estimate risk in select populations, particularly in younger individuals. A sizable number of people are also classified as being at intermediate risk, for whom the preventive strategy could be more precise. Also, there is substantial interest in improving existing risk models by incorporating nontraditional risk factors and, ultimately, reducing CVD events and mortality.

Personalized risk prediction, including Artificial Intelligence (AI) based analysis of coronary computed tomography angiography (CTA), coronary artery calcium scoring, electrocardiogram (ECG), risk factors, genomics, and metabolic risk scores may be able to improve risk assessment, pending supportive outcome data from clinical trials. A multidimensional approach to risk prediction holds the promise of precise risk prediction. This could allow for targeted prevention minimizing unnecessary costs and risks while optimizing outcomes. High-risk individuals could also be identified early in life, creating opportunities to arrest the development of nascent coronary atherosclerosis and prevent future clinical events.

In addition to risk prediction, AI-based analysis of imaging and biomarkers can be used to measure and predict progression and regression of disease, and its associated risk over time. Such a method to predict cardiovascular risk for a patient dynamically over time, given demographics, risk factors, genotype, phenotypes, and medications, can be used to identify effective preventive measures that can be taken to stop disease progression and to stabilize/regress existing disease. This approach of shifting from treating late-stage symptoms to proactively identifying early stages of heart disease and predicting and managing disease progression can result in improved patient outcomes as well as more cost-effective management of populations.

Consequently, the present disclosure describes new approaches for predicting the risk of developing cardiovascular disease as well as the risk of cardiovascular disease progression and/or regression in response to treatment and other factors.

It is to be understood that both the foregoing general description and the following detailed description are exemplary and explanatory only and are not restrictive of the disclosure.

### SUMMARY

According to certain aspects of the present disclosure, systems and methods are disclosed predicting cardiovascular disease risk.

In one embodiment, a computer-implemented method is disclosed for predicting cardiovascular disease risk, the method including: receiving a first patient history data comprising imaging data and/or non-imaging data for a patient at a first time point; selecting prediction report parameters defining a type of cardiovascular event and a risk prediction time scale; processing the first patient history data using a trained machine learning model configured to predict disease onset, progression, and/or regression over time, wherein the trained machine learning model is trained using patient subsets created based on patient history characteristics and outcomes; generating a risk prediction for the selected type of cardiovascular event over the selected risk prediction time scale based on the processed first patient history data; generating a risk prediction report based on the risk prediction; and outputting the risk prediction report.

In some aspects, the techniques described herein relate to a method, which may further include: receiving a second patient history data at a second time point; co-registering the second patient history data with the first patient history data; computing changes between the second patient history data at the second time point and the first patient history data at the first time point; generating an updated risk prediction based on the computed changes; generating an updated risk prediction report based on the updated risk prediction; and outputting the updated risk prediction report.

In other aspects, the techniques described herein relate to a method, wherein the imaging data may include coronary computed tomography angiography (CCTA) and/or non-enhanced cardiac computed tomography (NCCT).

In some aspects, the techniques described herein relate to a method, wherein the imaging data may include non-cardiac anatomy image data, including lung, carotids, peripherals, abdominal aorta, and/or retinal fundus.

In other aspects, the techniques described herein relate to a method, wherein the non-imaging data may include: risk factors (age, sex, high blood pressure, high low-density lipoprotein (LDL-H) cholesterol, diabetes, smoking and secondhand smoke exposure, obesity, unhealthy diet, and physical inactivity), blood markers, blood pressure measurements, body fat percentage or visceral body fat percentage, VO2 max, ECG, medication history, electronic medical record (EMR) information, invasive physiology measurements, and/or data from wearable and health monitoring devices.

In some aspects, the techniques described herein relate to a method, wherein the type of cardiovascular event may include an acute coronary syndrome event.

In other aspects, the techniques described herein relate to a method, wherein the risk prediction time scale may include multiple future time points.

In some aspects, the techniques described herein relate to a method, which may further include: receiving real-time monitoring data from monitoring systems; determining whether the real-time monitoring data indicates a change; updating the risk prediction based on the real-time monitoring data when a change is indicated; and generating a real-time alert when the updated risk prediction exceeds a predetermined alert threshold. In some aspects, the monitoring systems may include wearable devices.

In other aspects, the techniques described herein relate to a method, wherein the cardiovascular risk predictions may be adjusted based on patient intervention type and timing.

In some aspects, the techniques described herein relate to a method, wherein the prediction report parameters may further define prediction scales selected from lesion-level, vessel system-level, organ-level, and patient-level predictions.

In accordance with another embodiment, a system is disclosed for predicting cardiovascular disease risk, the system including: a processor configured to: receiving a first patient history data comprising imaging data and/or non-imaging data for a patient at a first time point; selecting prediction report parameters defining a type of cardiovascular event and a risk prediction time scale; processing the first patient history data using a trained machine learning model configured to predict disease onset, progression, and/or regression over time, wherein the trained machine learning model is trained using patient subsets created based on patient history characteristics and outcomes; generating a risk prediction for the selected type of cardiovascular event over the selected risk prediction time scale based on the processed patient history data; generating a risk prediction report based on the risk prediction; and outputting the risk prediction report.

In some aspects, the techniques described herein relate to a system, which may be further configured for: receiving a second patient history data at a second time point; co-registering the second patient history data with the first patient history data; computing changes between the second patient history data at the second time point and the first patient history data at the first time point; generating an updated risk prediction based on the computed changes; generating an updated risk prediction report based on the updated risk prediction; and outputting the updated risk prediction report.

In other aspects, the techniques described herein relate to a system, wherein the imaging data may include coronary computed tomography angiography (CCTA) and/or non-enhanced cardiac computed tomography (NCCT).

In some aspects, the techniques described herein relate to a system, wherein the non-imaging data may include: risk factors (age, sex, high blood pressure, high low-density lipoprotein (LDL-H) cholesterol, diabetes, smoking and secondhand smoke exposure, obesity, unhealthy diet, and physical inactivity), blood markers, blood pressure measurements, body fat percentage or visceral body fat percentage, VO2 max, ECG, medication history, electronic medical record (EMR) information, invasive physiology measurements, and/or data from wearable and health monitoring devices.

In other aspects, the techniques described herein relate to a system, wherein the risk prediction time scale may include multiple future time points.

In some aspects, the techniques described herein relate to a system, which may be further configured for: receiving real-time monitoring data from monitoring systems; determining whether the real-time monitoring data indicates a change; updating the risk prediction based on the real-time monitoring data when a change is indicated; and generating a real-time alert when the updated risk prediction exceeds a predetermined alert threshold.

In accordance with another embodiment, the techniques described herein relate to a non-transitory computer-readable medium storing instructions that, when executed by a computer, cause the computer to perform a method for predicting cardiovascular disease risk, the method including: receiving a first patient history data comprising imaging data and/or non-imaging data for a patient at a first time point; selecting prediction report parameters defining a type of cardiovascular event and a risk prediction time scale; processing the first patient history data using a trained machine learning model configured to predict disease onset, progression, and/or regression over time, wherein the trained machine learning model is trained using patient subsets created based on similar patient history characteristics and outcomes; generating a risk prediction for the selected type of cardiovascular event over the selected risk prediction time scale based on the processed first patient history data; generating a risk prediction report based on the risk prediction; and outputting the risk prediction report.

In some aspects, the techniques described herein relate to a non-transitory computer-readable medium, wherein the method may further include: receiving a second patient history data at a second time point; co-registering the second patient history data with the first patient history data; computing changes between the second patient history data at the second time point and the first patient history data at the first time point; generating an updated risk prediction based on the computed changes; generating an updated risk prediction report based on the updated risk prediction; and outputting the updated risk prediction report.

In some aspects, the techniques described herein relate to a non-transitory computer-readable medium, wherein the method may further include: receiving real-time monitoring data from monitoring systems; determining whether the real-time monitoring data indicates a change; updating the risk prediction based on the real-time monitoring data when a change is indicated; and generating a real-time alert when the updated risk prediction exceeds a predetermined alert threshold.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a block diagram depicting a network system for processing and sharing medical data.

FIG. 2 is a block diagram depicting a method for predicting cardiovascular disease risk and/or progression and/or regression.

FIG. 3 is a block diagram depicting a flowchart for training a machine learning model to predict CVD risk and/or risk of CVD progression and/or regression using patient data.

FIG. 4 is a block diagram depicting a method for predicting CVD risk and/or risk of CVD progression and/or regression using a patient's data and a trained machine learning model.

FIG. 5 is a block diagram depicting a flowchart for training a machine learning model to predict time to an acute coronary syndrome event using CCTA and ECG data.

FIG. 6 is a block diagram depicting a method for predicting time to an acute coronary syndrome event using a trained machine learning model and patient's CCTA and ECG data.

FIG. 7 is a block diagram depicting a flowchart for training a machine learning model to predict CVD risk and/or risk of CVD progression and/or regression using longitudinal patient data.

FIG. 8 is a block diagram depicting a method for updating CVD risk prediction and/or risk of CVD progression and/or regression based on a patient's longitudinal data.

FIG. 9 is a block diagram depicting a flowchart for training a machine learning model to predict risk of CVD and/or CVD progression and/or regression based on patient data and treatment.

FIG. 10 is a block diagram depicting a method for updating CVD risk prediction and/or risk of CVD progression and/or regression of a patient based on new treatment information.

FIG. 11 is a block diagram depicting a flowchart for training a machine learning model to predict CVD risk and/or risk of CVD progression and/or regression using patient data and real time monitoring data from wearable devices.

FIG. 12 is a block diagram depicting a method for updating CVD risk prediction and/or risk of CVD progression and/or regression using patient data and real-time monitoring data from wearable devices.

FIG. 13 is a block diagram depicting the architecture of a computer system for implementing the disclosed methods.

Notably, for simplicity and clarity of illustration, certain aspects of the figures depict the general configuration of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments.

### DETAILED DESCRIPTION

Reference will now be made in detail to the exemplary embodiments of the disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Techniques described in the current disclosure may utilize systems and methods described in U.S. application Ser. No. 14/011,151, U.S. application Ser. No. 15/680,950, U.S. application Ser. No. 16/226,204, U.S. application Ser. No. 18/483,759, and U.S. application Ser. No. 18/828,677 all of which are incorporated herein by reference.

As used herein, the term “exemplary” is used in the sense of “example,” rather than “ideal.” Moreover, the terms “a” and “an” herein do not denote a limitation of quantity but rather denote the presence of one or more of the referenced items.

The present disclosure is directed to a new approach for predicting the onset, progression, and regression of adverse cardiac events and for guiding medical treatment and other interventions based on patient-specific characteristics.

Risk assessment to optimize preventive strategies based on the absolute short- and long-term risk of an individual comprises the cornerstone of cardiovascular disease (CVD) prevention. The present disclosure, therefore, provides systems and methods for utilizing a patient's medical history, imaging data, biomarkers, and real-time monitoring information to generate accurate, personalized predictions of a patient's risk for developing CVD and/or the risk of CVD progression and/or regression across multiple time horizons. These predictions enable healthcare providers to implement targeted preventive measures and optimize patient-specific treatment strategies to improve clinical outcomes.

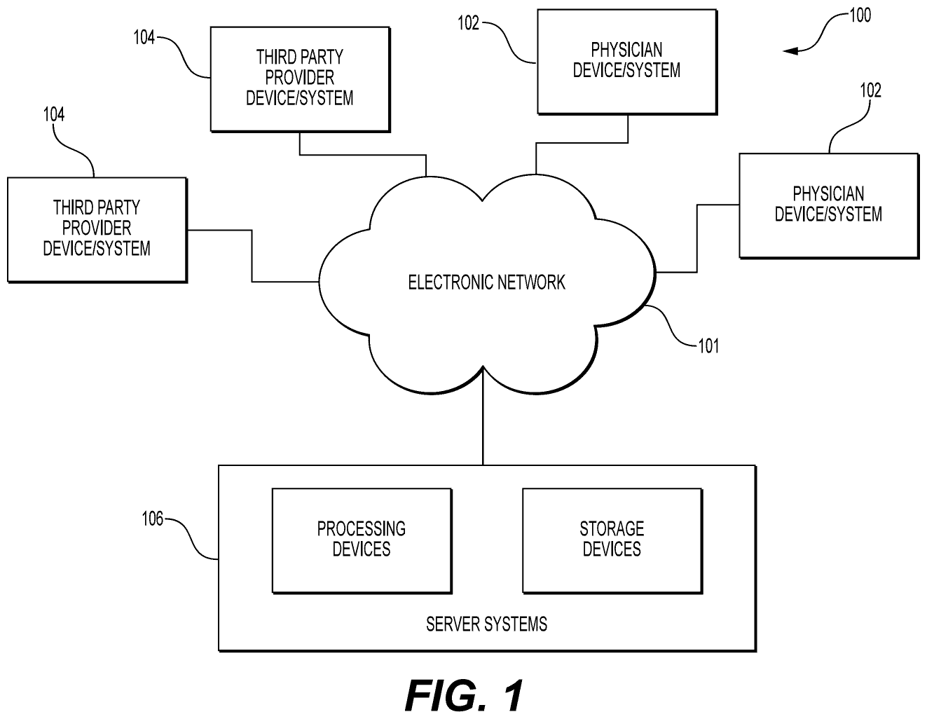

Referring now to the figures, FIG. 1 depicts a block diagram of an exemplary system and network for predicting the location, onset, and/or change of coronary lesions from vessel geometry, physiology, and hemodynamics. Specifically, FIG. 1 depicts a plurality of physician devices or systems 102 and third party provider devices or systems 104, any of which may be connected to an electronic network 101, such as the Internet, through one or more computers, servers, and/or handheld mobile devices. Physicians and/or third party providers associated with physician devices or systems 102 and/or third party provider devices or systems 104, respectively, may create or otherwise obtain images of one or more patients' cardiac and/or vascular systems. The physicians and/or third party providers may also obtain any combination of patient-specific information, such as age, medical history, blood pressure, blood viscosity, etc. Physicians and/or third party providers may transmit the cardiac/vascular images and/or patient-specific information to server systems 106 over the electronic network 101. Server systems 106 may include storage devices for storing images and data received from physician devices or systems 102 and/or third party provider devices or systems 104. Server systems 106 may also include processing devices for processing images and data stored in the storage devices.

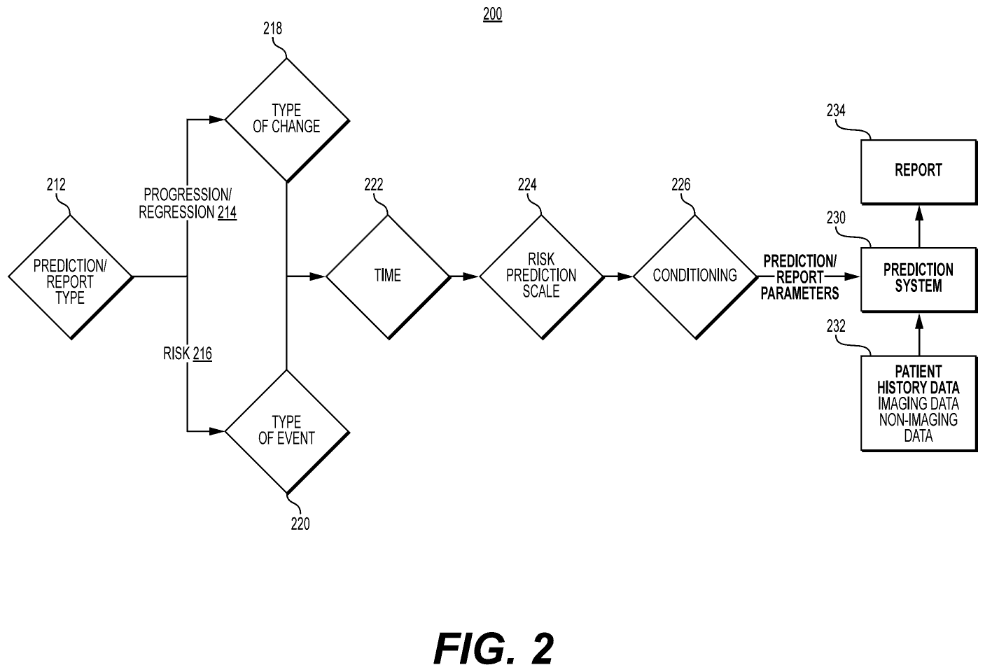

FIG. 2 is a block diagram of an exemplary method 200 for predicting cardiovascular disease risk and progression according to exemplary techniques of the present disclosure. As shown in FIG. 2, method 200 begins with a prediction/report type 212 selection, which branches into either a prediction of progression and/or regression of CVD 214 or a prediction of the risk of onset of CVD 216. When predicting the risk of onset of CVD 216, the system may require specification of a type of event 220, which may include: major adverse cardiovascular event (MACE), encompassing a composite of cardiovascular death, myocardial infarction, stroke, and urgent revascularization; mortality, which can be further categorized as all-cause mortality, cardiovascular mortality, or cause-specific mortality; stable chest pain, which may be classified according to Canadian Cardiovascular Society grading or other clinical classification systems; late revascularization or urgent revascularization; hospitalization, in general or for specific causes; development of specific cardiovascular disease conditions such as heart failure or hypertrophy; and presence of different types of cardiac and non-cardiac diseases, including all non-communicable diseases (such as coronary artery disease, heart failure, and peripheral artery disease), autoimmune diseases (such as rheumatoid arthritis and systemic lupus erythematosus), inflammatory diseases (such as inflammatory bowel disease), neurological diseases (such as Alzheimer's disease and Parkinson's disease), and metabolic diseases (such as diabetes mellitus and metabolic syndrome).

For progression and/or regression prediction 214, users may select the type of change 218 they want the system to predict, which may include changes in: lumen anatomy (e.g., % stenosis, minimum lumen diameter, lumen area, remodeling index); plaque parameters (e.g., total plaque volume, plaque composition including lipid-rich necrotic core, fibrous tissue, calcification, intraplaque hemorrhage, and high-risk plaque features such as positive remodeling, spotty calcification, napkin-ring sign, and low attenuation plaque); hemodynamic parameters (e.g., fractional flow reserve computed tomography (FFRct) and delta FFRct, coronary flow reserve (CFR), index of microcirculatory resistance (IMR), and other pressure-flow indices); ECG measurements (including ST-segment changes, T-wave morphology, QT interval, and other depolarization and repolarization parameters); heart structure and function (e.g., contractility, wall motion abnormalities, wall thickness, chamber dimensions); ejection fraction (including left ventricular ejection fraction, right ventricular ejection fraction, and changes in these parameters over time); risk factors (e.g., changes in low density lipoprotein-cholesterol (LDL-C), high-density lipoprotein cholesterol (HDL-C), triglycerides, blood pressure (BP), glycemic control, and inflammatory markers); pericoronary adipose tissue attenuation (which serves as a marker of coronary inflammation); epicardial adipose tissue volume or attenuation (which may indicate cardiovascular risk); and quality of life metrics (including physical functioning, emotional well-being, and disease-specific quality of life measures).

Users may specify a time 222 parameter indicating when the prediction is desired-either at a specific future time point (such as 1-year, 5-year, or 10-year risk) or across multiple time points to generate a time-to-event curve that provides a continuous risk assessment over an extended period.

Users may also select a risk prediction scale 224 from options including lesion-level predictions (focusing on specific coronary plaques or stenoses), vessel system-level predictions (analyzing the entire coronary tree or specific coronary arteries), organ-level predictions (assessing the heart as a whole or specific cardiac chambers), and patient-level predictions (evaluating overall cardiovascular risk for the individual).

Furthermore, at conditions 226, users may specify factors that potentially impact the prediction, such as: population averages (allowing for comparison with demographic-matched controls), medication regimens (including statins, antihypertensives, antiplatelets, anticoagulants, and novel cardioprotective agents), lifestyle and environmental factors (such as diet, physical activity, smoking status, alcohol consumption, and air pollution exposure), treatments (including percutaneous coronary intervention, coronary artery bypass grafting, and other interventional or surgical procedures), and health monitoring data from wearable devices (such as heart rate, physical activity levels, sleep patterns, and blood pressure measurements).

All of these parameters may collectively form the prediction report parameters 228 that are input into the machine learning prediction system 230. The machine learning prediction system 230 processes these parameters along with the patient history data 232 to generate a report 234 containing the requested risk prediction information. The report 234 may provide a prediction of risk of CVD onset and/or it may predict CVD progression and/or regression in terms of the expected longitudinal change and/or rate of change of risk factors such as lumen and plaque parameters, for example.

The machine learning prediction system 230 may be trained to utilize diverse patient history data 232, including cardiac imaging data such as: Coronary computed tomography angiography (CCTA) and/or non-enhanced cardiac computed tomography (NCCT), potentially with improved acquisition protocols specifically optimized for better risk or progression prediction, including higher temporal resolution, reduced motion artifacts, and enhanced plaque characterization; CCTA/NCCT-derived data including hemodynamic forces (such as endothelial shear stress, oscillatory shear index, and pressure gradients), anatomical features (including vessel tortuosity, bifurcation angles, and vessel tapering), plaque characteristics (such as plaque burden, composition, distribution, and vulnerability features), and myocardium at risk (MAR) features (quantifying the amount of myocardial tissue potentially affected by coronary stenosis); qualitative, semi-quantitative and quantitative perfusion imaging tests from various modalities (Positron Emission Tomography (PET), Single Photon Emission Computed Tomography (SPECT), Cardiovascular magnetic resonance (CMR), computed tomography perfusion (CTP)), including whole-heart or left ventricular (LV) myocardial blood flow (MBF) at rest and/or stress (measured in ml/min/g), regional (e.g., American Heart Association (AHA) segment level or territory level) MBF at rest and/or stress, whole-heart or regional myocardial blood flow reserve (MBFR) data (calculated as the ratio of stress to rest MBF); semi-quantitative metrics such as summed rest score, summed stress score, or summed difference score as derived for example from SPECT, Raw data (including projection data, k-space data, or sinogram data that can be processed with advanced reconstruction algorithms); CCTA+MBF (CT-derived dilation capacity or microvascular resistance reserve (MRRct), providing insights into both epicardial and microvascular function); 4-dimensional (4D) (Cine) CCTA (capturing coronary artery motion throughout the cardiac cycle); Cine CMR (providing detailed assessment of cardiac wall motion and function); 3-dimensional (3D)+time or 2-dimensional (2D)+time ECG (capturing spatial and temporal electrical activity of the heart); magnetic resonance angiography (MRA, offering non-contrast assessment of vascular structures); T1-weighted CMR (for tissue characterization, including assessment of fibrosis and edema), CMR elastography (measuring myocardial stiffness), 3D+time tagged CMR (for precise myocardial strain analysis); data from invasive imaging techniques including angiogram (providing luminography of coronary arteries), optical coherence tomography (OCT, offering high-resolution intravascular imaging), intravascular ultrasound (IVUS, providing cross-sectional vessel wall imaging), near infra-red spectroscopy (NIRS, for lipid core detection), and other invasive imaging modalities (such as angioscopy, photoacoustic imaging, and intravascular magnetic resonance imaging).

Additionally, non-cardiac imaging data may also be incorporated into the patient history data 232 for analysis by the machine learning prediction system 230. For example, non-cardiac organs, such as the lung, carotids, peripherals, abdominal aorta, and/or the retinal fundus, among other organs, may be imaged by means of the modalities described above or with other imaging modalities. These non-cardiac imaging data may provide information about systemic vascular health that may influence cardiovascular risk predictions generated by the machine learning prediction system 230. Specifically, lung imaging can reveal pulmonary vascular patterns, emphysema, and fibrosis that correlate with cardiovascular outcomes; carotid imaging can detect atherosclerotic plaque burden and stenosis that indicate systemic atherosclerosis; peripheral vascular imaging can identify peripheral artery disease which shares pathophysiological mechanisms with coronary artery disease; abdominal aortic imaging can detect aneurysms and calcification that reflect overall vascular health; and retinal fundus imaging can visualize microvascular changes that mirror coronary microvascular dysfunction.

Non-imaging data may also form a component of the patient history data 232 input to the machine learning prediction system 230. Examples of non-imaging patient data may include, among other things: Risk factors (age, sex, high blood pressure, high low-density lipoprotein (LDL-H) cholesterol, diabetes, smoking and secondhand smoke exposure, obesity, unhealthy diet, and physical inactivity); Blood markers (LDL-C, LDL-H, triglycerides, lipoprotein (a) (Lp(a)), apolipoprotein B (ApoB), high sensitivity C-reactive protein (hsCRP), troponins, B-type natriuretic peptide (BNP) and other biomarkers); genetics and proteomics data (polygenic risk scores, specific protein markers, etc.); blood pressure measurements; body fat percentage or visceral body fat percentage; VO2 max; ECG, including changes in ECG characteristics due to ischemia; medication history, such as the use of statins, anti-platelets, anticoagulants, GLP-1 agonists, SGLT2 inhibitors, and angiotensin II receptor blockers (ARBs); electronic medical record (EMR) information, including events, diagnoses, symptoms, treatments, and other tests; invasive physiology measurements, including but not limited to: fractional flow reserve (FFR), index of microvascular resistance (IMR), hyperemic microvascular resistance (hMR), microvascular resistance reserve (MRR), coronary flow reserve (CFR), instantaneous wave-free ratio (iFR)); and data from wearables and other health monitoring devices, including among other things, heart rate, respiratory rate, temperature, movement, sleep quality/quantity, blood oxygenation, and blood glucose. All of these data elements may be processed by the machine learning prediction system 230 to generate the report 234.

The imaging and non-imaging data used to construct patient history data 232 may be acquired over multiple time points, allowing the machine learning prediction system 230 to analyze temporal changes and trends when generating the report 234. For example, the system may analyze sequential CCTA scans taken at intervals of 3 months, 6 months, 1 year, or other clinically relevant timeframes to quantify changes in plaque volume, composition, and distribution. The system may track the transition of non-calcified plaque to calcified plaque, changes in positive remodeling indices, or the development of high-risk plaque features such as napkin-ring sign or low-attenuation plaque. Similarly, for non-imaging data, the system may incorporate serial measurements of biomarkers such as high-sensitivity C-reactive protein (hsCRP), troponin levels, or lipid profiles to identify biochemical trends that precede anatomical changes. This temporal analysis capability may enable the system to distinguish between stable disease patterns and those exhibiting significant progression or regression, which may impact risk stratification and treatment decisions. Furthermore, the system may correlate temporal changes with specific interventions or treatments, providing insights into treatment efficacy and patient-specific responses that may inform personalized therapeutic strategies detailed in the report 234.

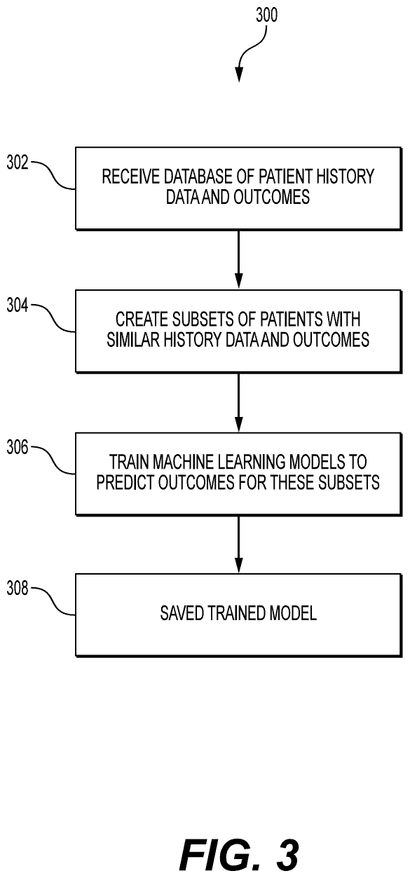

In some techniques, a machine learning prediction system 230 may be trained to provide CVD risk prediction and/or a prediction of CVD progression and/or regression. The process of training the machine learning system may include, as illustrated in FIG. 3, receiving a database of patient data and outcomes (events or progression/regression) in step 302, creating subsets of the patients that have similar history data and outcomes in step 304, and training the machine learning prediction system 230 to predict the outcomes of a subset of these patients in step 306. The patient data and outcomes database may include information relating to cardiac imaging data, non-cardiac imaging data, and non-imaging data. The outcomes database may be structured to enable time-to-event analysis and may include time-to-event durations from an initial scan to the first or multiple occurrences of the event for each type of event to be predicted. The outcomes database may also include durations to occurrence of medical procedures or timelines associated with adherence to medications. The complexity of the machine learning prediction system 230 may depend on the amount of data available, with deep learning architectures being employed when larger datasets are accessible. These models can incorporate various algorithms including a hierarchical deep learning architecture designed to model the relationship between the longitudinal multi-modal input data and time-to-event outcomes. This system may be composed of lesion-level, vessel-level, and patient-level feature encoders, which may be optionally pre-trained models using contrastive learning or masked autoencoder approaches, all of which may feed into patient-level aggregation modules and a final time-to-event prediction head. The prediction model may be trained end-to-end using survival analysis methodologies that account for the censoring of patient event durations. In addition to time-to-event prediction models, additional classes of models may be used in the prediction of progression and regression of the parameters described FIG. 2, using imaging and non-imaging patient data acquired at one or multiple time points. These may include deep learning models which are capable of capturing temporal dependencies and patterns from input data with a continuous time dimension (e.g. ECG and data from wearables), such as Long Short-Term Memory (LSTMs) or other recurrent neural networks (RNNs), gated recurrent unit (GRUs) and Transformers; models which can be trained to extract meaningful features from image data such as CNNs and Vision Transformers; models which are capable of handling heterogeneous data types and modalities, including Random Forests, multivariate regression models, multiple kernel learning, XGBoost, and multimodal deep learning models, for processing discrete and continuous data of various dimensionalities and formats (e.g., 1D-4D input signals, scalar variables such as risk factors, text descriptions, etc.); models which can learn rich, multimodal embeddings from combinations of input modalities, such as Vision-Language Models including CLIP, HLIP, Flamingo, as well as tabular-image models; large language models which can be used to extract and interpret text reports with relevant patient information, as well as be adapted in a multi-modal predictive model to formulate treatment recommendations and explain predictions; generative models which learn to synthesize and predict disease at future time points given inputs from one or more time points, including VAE, HVAE, VQ-GAN, residual diffusion models and latent diffusion models, in conjunction with auxiliary models and frameworks for modeling changes in relation to conditioning variables and time, such as ControlNet, deep structured causal models, and neural ODEs. Generative models may be applied to synthesize changes to the disease extent directly in the image data, from which derived parameters can be extracted using existing algorithms (e.g. quantification of plaque, as well as computation of FFRct). The machine learning system may involve training of multiple machine learning models described above, either on unimodal or multimodal input data to predict one or more of the target parameters. All such models may feed into one or more downstream machine learning models which aggregate features or predictions from upstream machine learning models for the prediction of one or more of the target parameters, including progression and/or regression as well as time-to-event predictions.

The patient subsets created in step 304 may be formed based on multiple clinical and demographic factors to enhance prediction accuracy for specific population segments. These factors may include age ranges, sex, comorbidity profiles, genetic markers, and baseline cardiovascular health status. For each subset, the system may employ different feature selection techniques to identify the relevant predictors for that particular patient group. This approach may allow the system to develop specialized prediction models tailored to distinct patient populations, thereby improving overall prediction accuracy compared to one-size-fits-all approaches. In step 308, the trained model may be saved for future use in predicting risk of CVD onset and/or progression/regression.

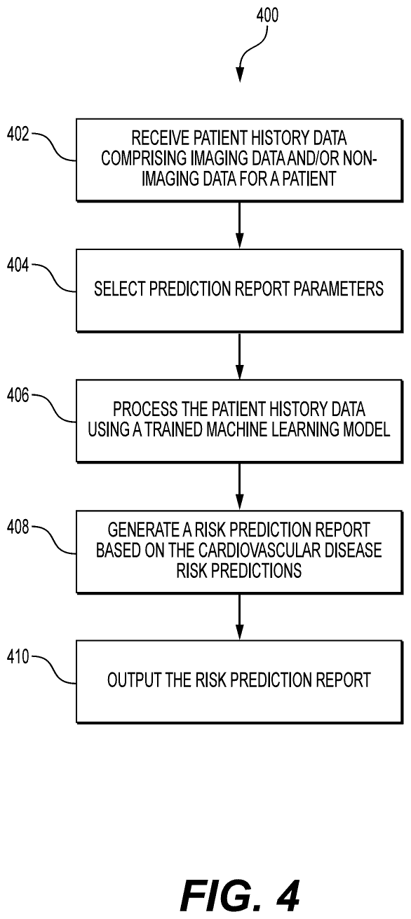

After the training of the machine learning system, it may be utilized to predict risk of CVD onset and/or progression/regression in clinical settings. As shown in FIG. 4, the method 400 for predicting cardiovascular disease risk begins with step 402, where the user may provide patient history data 232 to the machine learning prediction system 230 by uploading the data through a graphical user interface (GUI) or a digital imaging and communication in medicine (DICOM) connection. After uploading patient history data 232, in step 404, the user may be able to select the type of risk prediction they desire to receive through a GUI by either selecting reporting options from the prediction report parameters 228 discussed earlier. The trained machine learning system of FIG. 3 may then process the patient history data 232 in step 406, generate CVD risk predictions in step 408, and create a risk prediction report 234 in step 410. Finally, as depicted in step 412 of FIG. 4, the user may be able to download a prediction report 234 or select to upload this report to the electronic medical record, facilitating seamless integration with existing healthcare information systems.

The processing of patient history data 232 in step 406 may involve several computational techniques to extract meaningful features from the provided information. For imaging data such as CCTA, the system may employ image processing algorithms to identify relevant anatomical structures, quantify vessel stenosis, characterize plaque composition, and quantify FFRCT and percent myocardial blood flow. The aforementioned derived features may be obtained by means of a pipeline of algorithms that reconstruct the coronary tree geometry, including lumen and outer wall, and may solve a physiological simulation of pressure and flow in the reconstructed geometry. Other image-derived features may be learned or extracted from various representations, including 2D and 3D images or image patches, optionally derived from regions of interest in the image data, such as the coronary tree from CCTA, or the myocardium from CCTA, CMR, PET, SPECT, etc. Learning of image-based features from these representations can be performed using the relevant models described in FIG. 3. Furthermore, non-imaging data, including blood markers, medication history, and demographic information, may be normalized and standardized to ensure consistent analysis across different measurement scales. Data imputation techniques may be used to process missing patient data that may be required by the machine learning system.

The risk prediction report 234 generated in step 410 may provide information tailored to the specific needs of healthcare providers and patients. The report 234 may include quantitative risk scores with associated confidence intervals, visual representations of risk trajectories over time 222 and comparative analyses showing how the patient's risk profile compares to demographic-matched population averages. Additionally, the report 234 may highlight the specific risk factors that contribute to the patient's cardiovascular risk, enabling targeted intervention strategies. For progression and/or regression predictions, the report 234 may include predicted changes of quantitative parameters of lumen and plaque at selected scales (patient, vessel, segment, lesion) such as maximum percent lumen stenosis, plaque volumes, and presence of high risk plaque features, including comparative analyses of these disease progression or regression markers with respect to change trajectories derived from a demographic-matched population. The report may also include visualizations indicating which part of the coronary anatomy (e.g. vessel, segment, or lesion) is predicted to observe significant changes, as well as detailed visualizations of the predicted anatomical and physiological changes over time 222, allowing clinicians to anticipate disease evolution and adjust treatment plans accordingly. The integration with electronic medical records in step 412 ensures that these predictions become part of the patient's longitudinal health record, enabling continuous risk monitoring and facilitating coordinated care across different healthcare providers.

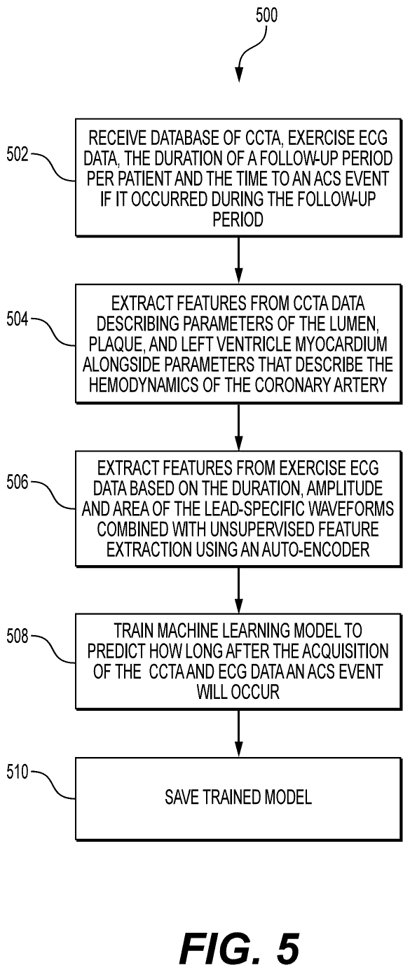

In another technique, a machine learning system may be trained to predict time to an acute coronary syndrome (ACS) event given CCTA and exercise ECG data. As illustrated in FIG. 5, the method 500 begins with step 502, where the system receives a database of CCTA data, exercise ECG data, the duration of a follow-up period per patient data, and data regarding the time to an ACS event if it occurred during the follow-up period. In step 504, features may be extracted from CCTA data describing parameters of the lumen, plaque, and left ventricle myocardium alongside parameters that describe the hemodynamics of the coronary artery. In step 506, features may be extracted from exercise ECG data based on the duration, amplitude and area of the lead-specific waveforms combined with unsupervised feature extraction using an auto-encoder. At step 508, a machine learning model may be trained to predict how long after the acquisition of the CCTA and ECG data an ACS event will occur, for example with survival analysis models described in FIG. 3. In step 510, the trained model may be saved for future use in predicting the time to potential ACS events in patients.

The feature extraction process in steps 504 and 506 may involve computational techniques to extract features from: lumen and plaque images, including anatomical modeling using deep learning models for vessel center localization followed by cross-sectional lumen and outer vessel wall and/or plaque contouring, plaque composition characterization and quantification, percent stenosis calculation based on a fitted model of healthy lumen, e.g., utilizing the plaque segmentation, and positive remodeling indices calculated from predicted outer wall boundary and model of healthy lumen; images of LV myocardium and heart chambers, including LV myocardium shape, mass and motion estimation, left atrial volume and left atrial appendage volume, enlargement of any heart chamber, presence of hypertrophy or amyloidosis, and ejection fraction; valves and aorta images, including calcification, stenosis or inflammation of the valves, enlargement, dissection, aneurysm or stenosis of the aorta; hemodynamics, including changes in FFRct across lesions and distribution of FFRct in coronary arteries, and myocardial mass subtended at locations of focal lesions; radiomic properties of perivascular and epicardial adipose tissue, including attenuation values; and direct image data from volumes or slices. These methods may also incorporate techniques discussed in U.S. application Ser. No. 15/975,197, filed May 9, 2018, U.S. application Ser. No. 15/852,119, filed Dec. 22, 2017, and U.S. application Ser. No. 13/013,561, filed Jan. 25, 2011, which are incorporated herein by reference.

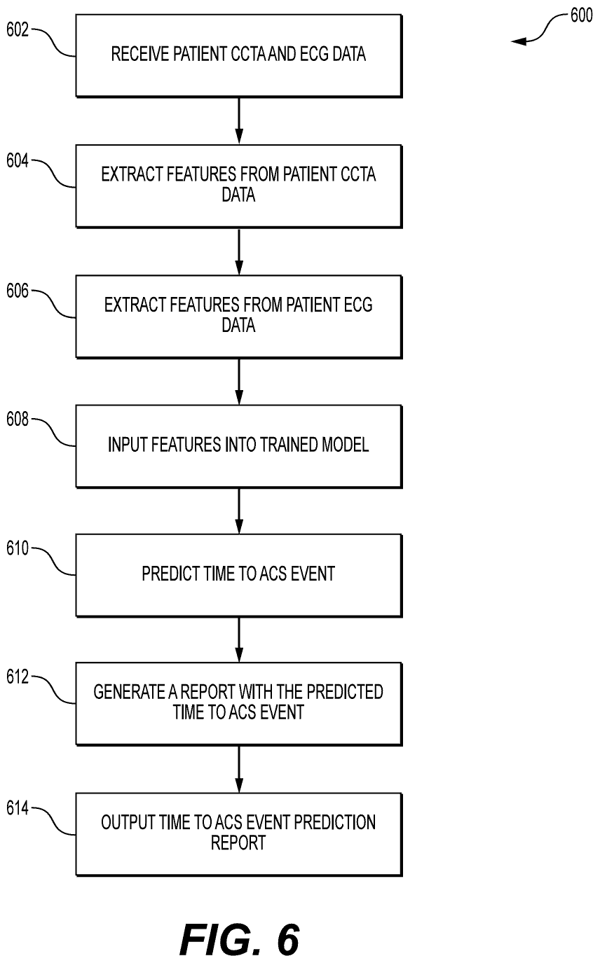

For implementing predictions using the trained model, as shown in FIG. 6, the method 600 begins with step 602, where patient CCTA and ECG data are received. The method 600 then moves to step 604, where features are extracted from the patient CCTA data, followed by step 606, where features may be extracted from the patient ECG data. In step 608, these extracted features are input into the trained model of FIG. 5. The method 600 then advances to step 610, where the time to ACS event may be predicted based on the processed features. In step 612, a report may be generated with the predicted time to ACS event. In step 614, the time to ACS event prediction report may be outputted and/or saved. The user may download the report, view an updated risk prediction on the portal/website, and/or upload results to the electronic medical record for a report.

The prediction report generated in step 612 may contain multiple elements designed to provide insights to inform clinical decisions or clinical decision support. These elements may include a numerical estimate of time to potential ACS event with associated confidence intervals, a risk stratification category (e.g., high, intermediate, or low risk), and a visual representation of the prediction timeline. The report may visualize what aspects of the patient's anatomy, coronary arteries, heart, or other structures contribute to the risk prediction. The report may also highlight the specific imaging and ECG features that influenced the prediction, providing clinicians with actionable insights for targeted interventions. For instance, if the model identified vulnerable plaque characteristics or specific ECG abnormalities as primary contributors to elevated risk, these would be displayed. The system may also provide comparative analyses showing how the patient's risk profile compares to population averages or similar demographic groups. Additionally, the report could include simulation results showing how potential interventions (such as statin therapy, antiplatelet medications, or lifestyle modifications) might alter the predicted timeline, enabling physicians to engage in data-driven shared decision-making with patients regarding preventive strategies. The electronic medical record integration capability ensures that this predictive information becomes part of the patient's longitudinal health record, facilitating ongoing risk monitoring and treatment planning. The report may directly suggest guidelines or recommendations for treatment plans based on the level of risk, for example, if a patient has the highest level of risk for an adverse coronary event, the report may reference guidelines or clinical studies to recommend a corresponding level of medical therapy for high-risk patients.

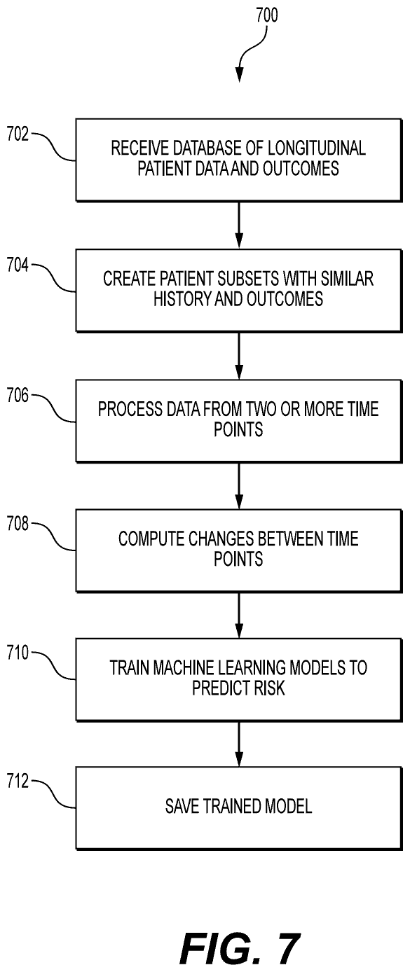

In an alternative technique, a machine learning system may be trained for risk prediction based on an initial time point, as described in FIG. 4, and the updated with additional data from multiple time points to both understand how the disease has progressed and also provide an updated risk assessment. As illustrated in FIG. 7, the method 700 for training such a machine learning model begins with step 702, where a database of longitudinal patient data and outcomes (events or progression/regression) is received. The process then moves to step 704, where patient subsets with similar patient data and outcomes may be created to enable model training. These patient subsets may be formed based on multiple clinical and demographic factors, including age ranges, sex, comorbidity profiles, genetic markers, and baseline cardiovascular health status, allowing the system to develop specialized prediction models tailored to distinct patient populations.

Following the creation of patient subsets, in step 706, the system may process data from two or more time points for each patient, establishing the temporal relationships for progression and/or regression analysis. This temporal processing may involve image registration techniques to align anatomical structures across different imaging sessions, normalization of biomarker measurements to account for inter-test variability, and standardization of clinical assessments to ensure consistent evaluation across time points. In step 708, the system may compute changes between these time points, extracting progression and/or regression patterns. These computations may include, among other things, quantification of plaque volume changes, alterations in stenosis percentage, shifts in plaque composition from non-calcified to calcified components, changes in hemodynamic parameters such as fractional flow reserve, and variations in biomarker levels that indicate disease activity. The method 700 continues to step 710, where machine learning models may be trained to predict outcomes based on these computed changes, and concludes with step 712, where the trained model may be saved for future use in updating a patient's risk of CVD and/or CVD progression/regression. The complexity of the machine learning models may depend on the amount of data available. Training step 710 may include one or more of the methods described in FIG. 3 and associated specification text. For example, training a deep neural network which incorporates inputs from multiple time-points, along with the changes between those time points and duration between time-points to predict regression and progression of each parameter at a specified future time. The deep neural network may ingest three vectors, containing the derived parameters described above at each time point and the difference in parameters between the two time-points, respectively.

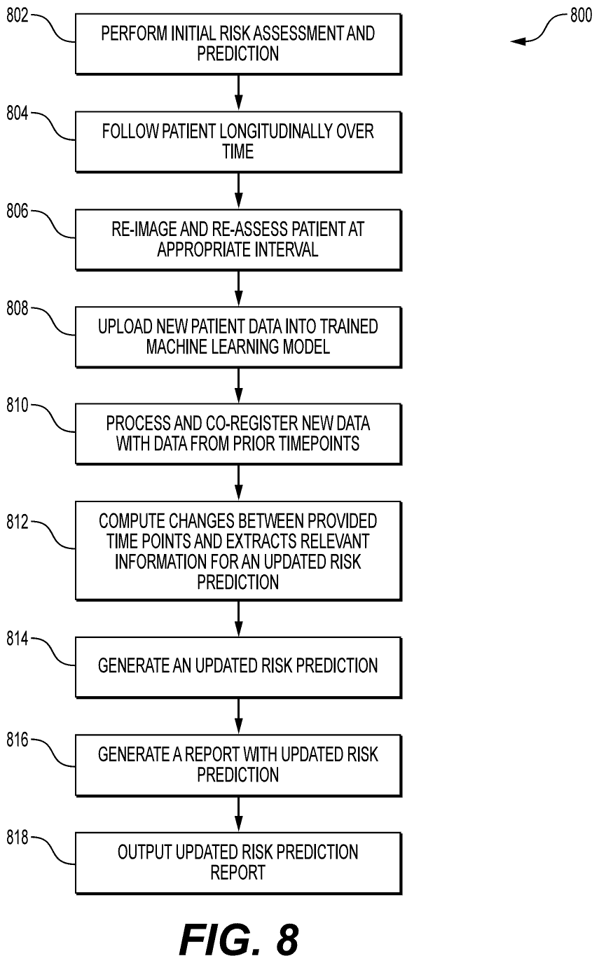

After the initial training, the trained model in FIG. 7 can be applied in a workflow as depicted in FIG. 8. The method 800 begins with step 802, where an initial risk assessment and prediction may be performed for a patient as shown and discussed in FIG. 4. In step 804, the patient may be followed over time, and at step 806, the patient may be re-imaged and re-assessed at appropriate time intervals. As shown in step 808, the user may upload new data (e.g., new imaging or other data) into the trained machine learning model described in FIG. 7 and associated specification text. The system may then process the new data in step 810 and may co-register the information to any prior time points of the patient risk assessment. In step 812, the system may compute changes between provided time points and may extract the relevant information for an updated risk prediction. These computations may include not only direct measurements of anatomical changes but also derived metrics such as progression rates, acceleration or deceleration of disease activity, and response patterns to interventions or treatments. The process advances to step 814, where an updated risk prediction may be generated based on the analyzed longitudinal data. In step 816, a report may be generated with the updated risk prediction. In step 818, the system may output the updated risk report. The user can download the report, view an updated risk prediction on the portal/website, and/or upload results to the electronic medical record for a report indicating the impact on risk prediction of specified interventions and durations, relative to previous predictions.

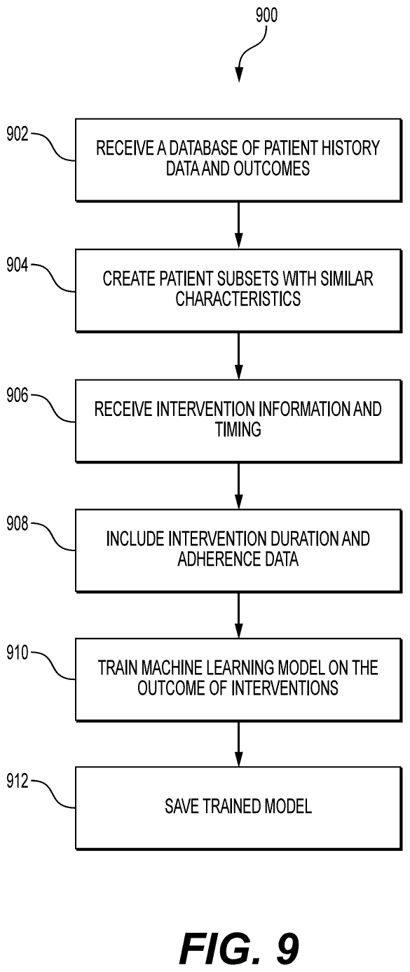

In another technique, a machine learning system may be trained for risk prediction, using information provided for an initial risk assessment, e.g., with imaging data and non-imaging data, together with information about planned or completed interventions and their durations. As illustrated in FIG. 9, method 900 for training such a machine learning system begins with step 902, where a database of patient data (including imaging and non-imaging data) and outcomes (events or progression/regression) may be received. The process then moves to step 904, where subsets of patients with similar history data and outcomes may be created. These patient subsets may be formed based on multiple clinical and demographic factors, including age ranges, sex, comorbidity profiles, genetic markers, and baseline cardiovascular health status. Training steps for the machine learning system may involve a multi-modal deep learning model, with a MLP encoder for the non-imaging data in tabular format, and a CNN or ViT encoder for the image data. The tabular data can include information about interventions and their durations. The model can be trained on the outcomes of patients for whom interventions, durations and outcomes are available, with a survival loss for time-to-event prediction and a prediction head for regression/progression of target parameters such as plaque volume in an end-to-end fashion. The image encoder and tabular encoder may be pre-trained on a larger dataset without outcomes data, as a way to mitigate overfitting with limited training data with outcomes.

Following this, in step 906, intervention information and timing are received, indicating when each type of intervention was started in relation to imaging or other collected patient data. This intervention information may include, but is not limited to: (1) Medical treatments such as detailed medication data (including drug class, specific medication, dosage, frequency, and administration route), interventional procedures (including percutaneous coronary intervention, coronary artery bypass grafting, valve replacement, or implantable device placement), and advanced therapies (such as gene therapy, stem cell treatments, or novel biologics); (2) Lifestyle modifications including dietary changes (such as Mediterranean diet adoption, sodium restriction, or plant-based eating patterns), structured exercise regimens (including aerobic, resistance, or combined training programs), smoking cessation programs, stress management techniques (such as mindfulness meditation, cognitive behavioral therapy, or biofeedback), weight management interventions, and sleep optimization strategies; and/or (3) Other interventions including cardiac rehabilitation programs, remote patient monitoring systems, digital health applications, educational interventions, social support networks, environmental modifications, and complementary approaches (such as acupuncture or yoga) when used as adjunctive therapies. At step 908, intervention duration and adherence data may also be included in the analysis. The adherence data may be quantified through medication possession ratios, proportion of days covered, electronic monitoring systems, wearable device data tracking lifestyle adherence, digital health application engagement metrics, or patient-reported adherence scales.

In step 910, a machine learning model may be trained to learn the impact of these interventions on outcomes. The model may incorporate time-dependent variables to account for the dynamic nature of intervention effects, allowing for the analysis of both immediate and delayed impacts of interventions on cardiovascular risk. Additionally, the model may be designed to identify potential intervention interactions, where the effect of one approach may be enhanced or diminished by the presence of another intervention. For example, the model might detect synergistic effects between specific medications and specific exercise regimens or identify how dietary patterns might influence medication efficacy. The model may also account for the sequencing of interventions, recognizing that the order in which treatments, lifestyle changes, and other interventions are implemented may affect their cumulative impact. Finally, in step 912, the trained model may be saved for future use, with appropriate version control to ensure reproducibility and facilitate model updates as new data becomes available. The type of machine learning model(s) may include deep structured causal models capable of modeling interdependencies of causal variables including interventions and time of different events on imaging data and other relevant formats. Alternatively, training deep generative models such as VQ-GAN combined with a ControlNet or Neural ODE could be used to model the effects of multiple events and interventions over time to be modeled in relation to progression of disease.

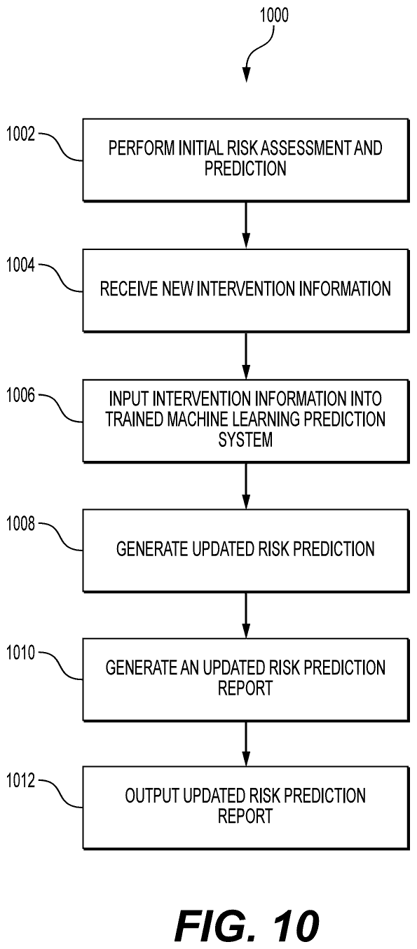

After the machine learning model is trained, it can be applied to provide updated risk of CVD and/or CVD progression/regression predictions with additional intervention data. As shown in FIG. 10, the method 1000 begins with step 1002, where an initial risk assessment and prediction is performed for a patient, as shown in FIG. 4. In step 1004, new intervention information may be received from the patient or healthcare provider regarding changes in treatment approaches. This information may include, but is not limited to: (1) Medical treatment modifications such as medication initiation, discontinuation, dose adjustments, adherence changes, or switches between therapeutic agents, as well as scheduling of interventional procedures or changes to procedural plans; (2) Lifestyle intervention updates including changes in dietary patterns, exercise frequency or intensity, progress in smoking cessation efforts, adoption of stress management practices, or modifications to sleep hygiene approaches; and (3) Other intervention adjustments such as enrollment in cardiac rehabilitation, implementation of remote monitoring solutions, engagement with digital health platforms, participation in educational programs, or integration of complementary therapies. The system may also incorporate contextual information about the reasons for intervention changes, such as adverse effects, cost considerations, accessibility issues, patient preferences, or clinical response, which can provide valuable insights for the prediction model. In step 1006, this new data may be fed into the trained model of FIG. 9, where it undergoes preprocessing to standardize formats and address any missing values. The method 1000 then proceeds to step 1008, where an updated risk prediction may be generated based on the intervention information. This updated prediction may include not only revised risk estimates but also confidence intervals that reflect the certainty of the prediction based on the available evidence for similar intervention scenarios. In step 1010, an updated risk prediction report may be generated. The report may also include visualizations comparing the patient's predicted risk trajectory under different intervention scenarios, facilitating shared decision-making between healthcare providers and patients regarding therapeutic strategies that may combine medical treatments, lifestyle modifications, and other complementary approaches. In step 1012, the updated risk prediction report may be outputted. The user can download the report, view an updated risk prediction on the portal/website, or upload results to the electronic medical record for a report.

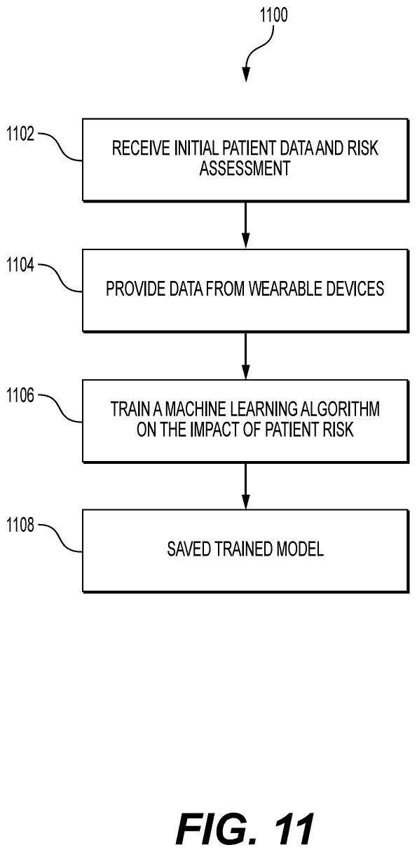

In another technique, the system may adjust risk prediction using real-time monitoring data. In this technique, a machine learning system may be trained for risk prediction, combining information from imaging data with non-imaging data, including continuous and real-time health information. As illustrated in FIG. 11, the method 1100 for training such a machine learning algorithm begins with step 1102, where initial patient data and risk assessment information are received. This includes receiving a database of patient history data (including imaging and non-imaging data) and outcomes (events or progression/regression) and creating subsets of the patients that have similar history data and outcomes. The initial patient data may include baseline assessments such as CCTA images, ECG recordings, laboratory biomarkers, and demographic information that establish the patient's cardiovascular status prior to continuous monitoring, as described earlier. Following this, in step 1104, data from wearable devices is provided to the system. This data may include heart rate variability metrics (including time-domain, frequency-domain, and non-linear measures), blood pressure, electrical resistance or impedance measurements, movement/exercise patterns (including step counts, activity intensity, and duration of sedentary periods), derived assessments such as stress levels, activity levels, exercise indices, respiratory rate fluctuations, temperature variations, continuous or intermittent blood glucose measurements, sleep architecture parameters (including rapid eye movement (REM) sleep percentage, deep sleep duration, and sleep fragmentation indices), or other health monitoring devices (e.g., pulse oximeter measurements showing oxygen saturation trends, single-lead or multi-lead ECG measurements capturing ST-segment changes, T-wave alterations, and arrhythmia episodes). The method 1100 then advances to step 1106, where a machine learning algorithm may be trained on the impact of these additional data streams on patient risk prediction. In step 1108, the trained model may be saved for future use in updating a patient's CVD risk and/or CVD progression or regression.

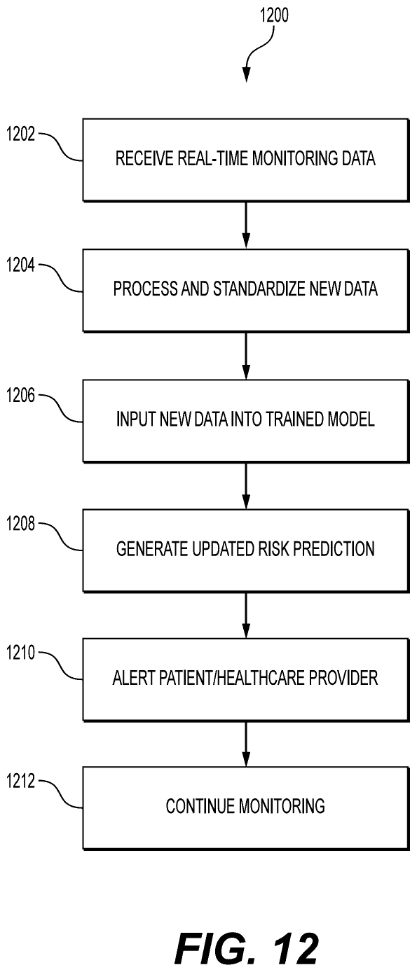

Following the training, the system may provide an updated prediction of a patient's CVD risk and/or CVD progression and/or regression with additional data input as depicted in FIG. 12. The method 1200 begins with step 1202, where real-time monitoring data may be received from wearable devices or other health monitoring activities. This data acquisition may occur through secure application programming interfaces (APIs) that connect to various wearable device platforms, direct Bluetooth or Wi-Fi connections to monitoring devices, or through patient-facing mobile applications that collect and transmit health data. The system may implement data validation protocols to identify and manage missing data, outliers, or device malfunctions that could affect prediction accuracy. In step 1204, this new data may be processed by analyzing the outputs directly, such as a patient's blood pressure or heart rate; applying machine learning models to extract insights from the data, such as analyzing an EKG or activity data to inform features that can be input into a risk model; or the raw data may be summarized by taking various measures such as mean, peak, or other values over a period of time. Multiple data sources may be combined into a machine learning model to inform the risk prediction. In step 1206, the processed data may be input into the trained model of FIG. 11. In step 1208, an updated risk prediction may be generated based on the processed monitoring data. In some instances, the updated risk prediction may be generated based on a change detected in real-time monitoring data. Furthermore, the updated risk prediction may be generated when a change exceeding a predetermined threshold is detected in the real time monitoring data. In some instances, a change in the real-time monitoring data exceeding a predetermined threshold level may be significant. This updated prediction may incorporate confidence intervals that reflect the certainty of the prediction based on the quality and quantity of the monitoring data available. The system may be authorized to receive data from wearable devices or other health monitoring devices in real-time, and to update its risk prediction based on this data. The authorization process may include secure patient consent mechanisms, compliance with healthcare data privacy regulations, and encryption protocols to protect sensitive health information during transmission and storage.

As shown in step 1210 of FIG. 12, the system may provide real-time alerts when provided data (such as an event picked up by live monitoring data) that alter risk prediction above a predetermined threshold level. The system may provide this alert to the patient and/or healthcare provider. The alerting mechanism may incorporate a tiered notification system based on the magnitude and urgency of the risk change—from routine updates for minor fluctuations to immediate high-priority alerts for substantial risk increases that may require prompt medical attention. These alerts may be delivered through multiple channels including mobile push notifications, text messages, email, or direct integration with electronic health record (EHR) systems for clinical workflow integration. The alert content may be tailored to the recipient, with patient-facing notifications focusing on actionable recommendations and healthcare provider alerts including more detailed clinical information and suggested intervention pathways. The user can download a report, view an updated risk prediction on the portal/website, or upload results to the electronic medical record for a report indicating the impact on risk prediction of specified data, relative to previous predictions. These reports may include visualizations such as trend graphs showing risk trajectory over time, contributing factor analyses highlighting which physiological parameters influenced the risk change, and comparative analyses showing how the patient's patterns compare to population norms or their own historical baselines. As depicted in step 1212, the monitoring may continue, allowing for ongoing risk assessment and timely interventions when necessary. The continuous monitoring system may incorporate adaptive sampling rates that increase data collection frequency during periods of detected physiological stress or abnormality, while conserving device battery and computational resources during periods of stability. The system may also implement periodic recalibration protocols to maintain prediction accuracy as the patient's baseline condition evolves over time.

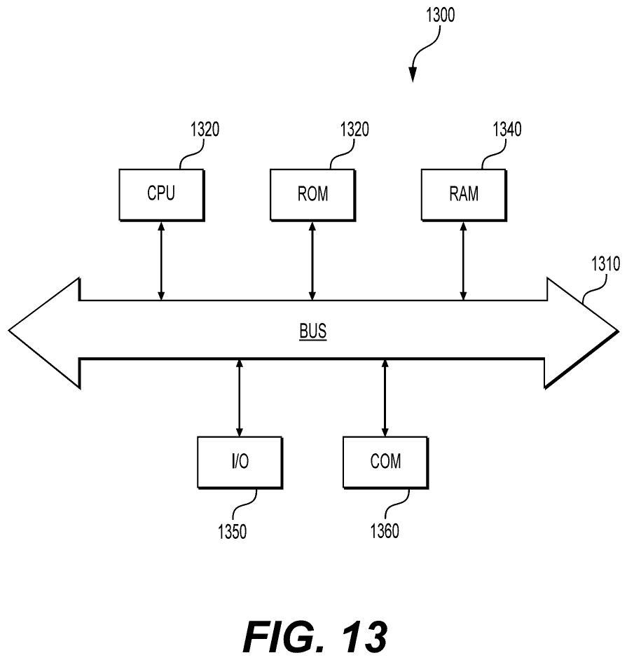

FIG. 13 is a simplified block diagram of an exemplary computer system 1300 in which embodiments of the present disclosure may be implemented, for example as any of the physician devices or servers 102, third party devices or servers 104, and server systems 106. A platform for a server 1300, for example, may include a data communication interface for packet data communication 1360. The platform may also include a central processing unit (CPU) 1320, in the form of one or more processors, for executing program instructions. The platform typically includes an internal communication bus 1310, program storage and data storage for various data files to be processed and/or communicated by the platform such as ROM 1330 and RAM 1340, although the server 1300 often receives programming and data via a communications network (not shown). The hardware elements, operating systems and programming languages of such equipment are conventional in nature, and it is presumed that those skilled in the art are adequately familiar therewith. The server 1300 also may include input and output ports 1350 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

As described above, the computer system 1300 may include any type or combination of computing systems, such as handheld devices, personal computers, servers, clustered computing machines, and/or cloud computing systems. In one embodiment, the computer system 1300 may be an assembly of hardware, including a memory, a central processing unit (“CPU”), and/or optionally a user interface. The memory may include any type of RAM or ROM embodied in a physical storage medium, such as magnetic storage including floppy disk, hard disk, or magnetic tape; semiconductor storage such as solid-state disk (SSD) or flash memory; optical disc storage; or magneto-optical disc storage. The CPU may include one or more processors for processing data according to instructions stored in the memory. The functions of the processor may be provided by a single dedicated processor or by a plurality of processors. Moreover, the processor may include, without limitation, digital signal processor (DSP) hardware, or any other hardware capable of executing software. The user interface may include any type or combination of input/output devices, such as a display monitor, touchpad, touchscreen, microphone, camera, keyboard, and/or mouse.

Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms, such as computer or machine “readable medium” refer to any medium that participates in providing instructions to a processor for execution.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A computer-implemented method for predicting cardiovascular disease risk, the method comprising:
receiving a first patient history data comprising imaging data and/or non-imaging data for a patient at a first time point;
selecting prediction report parameters defining a type of cardiovascular event and a risk prediction time scale;
processing the first patient history data using a trained machine learning model configured to predict disease onset, progression, and/or regression over time, wherein the trained machine learning model is trained using patient subsets created based on patient history characteristics and outcomes;
generating a risk prediction for the selected type of cardiovascular event over the selected risk prediction time scale based on the processed first patient history data;
generating a risk prediction report based on the risk prediction; and
outputting the risk prediction report.

2. The method of claim 1, further comprising:
receiving a second patient history data at a second time point;
co-registering the second patient history data with the first patient history data;
computing changes between the second patient history data at the second time point and the first patient history data at the first time point;
generating an updated risk prediction based on the computed changes;
generating an updated risk prediction report based on the updated risk prediction; and
outputting the updated risk prediction report.

3. The method of claim 1, wherein the imaging data comprises Coronary computed tomography angiography (CCTA) and/or non-enhanced cardiac computed tomography (NCCT).

4. The method of claim 1, wherein the imaging data comprises non-cardiac anatomy image data, including lung, carotids, peripherals, abdominal aorta, and/or retinal fundus.

5. The method of claim 1, wherein the non-imaging data comprise: risk factors (age, sex, high blood pressure, high low-density lipoprotein (LDL-H) cholesterol, diabetes, smoking and secondhand smoke exposure, obesity, unhealthy diet, and physical inactivity), blood markers, blood pressure measurements, body fat percentage or visceral body fat percentage, VO2 max, ECG, medication history, electronic medical record (EMR) information, invasive physiology measurements, and/or data from wearable and health monitoring devices.

6. The method of claim 1, wherein the type of cardiovascular event comprises an acute coronary syndrome event.

7. The method of claim 1, wherein the risk prediction time scale comprises multiple future time points.

8. The method of claim 1, further comprising:
receiving real-time monitoring data from monitoring systems;
determining whether the real-time monitoring data indicates a change;
updating the risk prediction based on the real-time monitoring data when a change is indicated; and
generating a real-time alert when the updated risk prediction exceeds a predetermined alert threshold.

9. The method of claim 8, wherein the monitoring systems comprise wearable devices.

10. The method of claim 1, wherein the cardiovascular risk predictions are adjusted based on patient intervention type and timing.

11. The method of claim 1, wherein the prediction report parameters further define prediction scales selected from lesion-level, vessel system-level, organ-level, and patient-level predictions.

12. A system for predicting cardiovascular disease risk, the system comprising:
a processor configured to: receiving a first patient history data comprising imaging data and/or non-imaging data for a patient at a first time point;
selecting prediction report parameters defining a type of cardiovascular event and a risk prediction time scale;
processing the first patient history data using a trained machine learning model configured to predict disease onset, progression, and/or regression over time, wherein the trained machine learning model is trained using patient subsets created based on patient history characteristics and outcomes;
generating a risk prediction for the selected type of cardiovascular event over the selected risk prediction time scale based on the processed patient history data;
generating a risk prediction report based on the risk prediction; and
outputting the risk prediction report.

13. The system of claim 12, further comprising:
receiving a second patient history data at a second time point;
co-registering the second patient history datawith the first patient history data;
computing changes between the second patient history data at the second time point and the first patient history data at the first time point;
generating an updated risk prediction based on the computed changes;
generating an updated risk prediction report based on the updated risk prediction; and
outputting the updated risk prediction report.

14. The system of claim 12, wherein the imaging data comprises Coronary computed tomography angiography (CCTA) and/or non-enhanced cardiac computed tomography (NCCT).

15. The system of claim 12, wherein the non-imaging data comprise: risk factors (age, sex, high blood pressure, high low-density lipoprotein (LDL-H) cholesterol, diabetes, smoking and secondhand smoke exposure, obesity, unhealthy diet, and physical inactivity), blood markers, blood pressure measurements, body fat percentage or visceral body fat percentage, VO2 max, ECG, medication history, electronic medical record (EMR) information, invasive physiology measurements, and/or data from wearable and health monitoring devices.

16. The system of claim 12, wherein the risk prediction time scale comprises multiple future time points.

17. The system of claim 12, further comprising:
receiving real-time monitoring data from monitoring systems;
determining whether the real-time monitoring data indicates a change;
updating the risk prediction based on the real-time monitoring data when a change is indicated; and
generating a real-time alert when the updated risk prediction exceeds a predetermined alert threshold.

18. A non-transitory computer-readable medium storing instructions that, when executed by a computer, cause the computer to perform a method for predicting cardiovascular disease risk, the method comprising:
receiving a first patient history data comprising imaging data and/or non-imaging data for a patient at a first time point;
selecting prediction report parameters defining a type of cardiovascular event and a risk prediction time scale;
processing the first patient history data using a trained machine learning model configured to predict disease onset, progression, and/or regression over time, wherein the trained machine learning model is trained using patient subsets created based on similar patient history characteristics and outcomes;
generating a risk prediction for the selected type of cardiovascular event over the selected risk prediction time scale based on the processed first patient history data;
generating a risk prediction report based on the risk prediction; and
outputting the risk prediction report.

19. The non-transitory computer-readable medium of claim 18, further comprising:
receiving a second patient history data at a second time point;
co-registering the second patient history data with the first patient history data;
computing changes between the second patient history data at the second time point and the first patient history data at the first time point;
generating an updated risk prediction based on the computed changes;
generating an updated risk prediction report based on the updated risk prediction; and
outputting the updated risk prediction report.

20. The non-transitory computer-readable medium of claim 18, further comprising:
receiving real-time monitoring data from monitoring systems;
determining whether the real-time monitoring data indicates a change;
updating the risk prediction based on the real-time monitoring data when a change is indicated; and
generating a real-time alert when the updated risk prediction exceeds a predetermined alert threshold.
